(19) United States
(12) Patent Application Publication
(10) Pub. No.: US 2018/0325452 A1
(43) Pub. Date: Nov. 15, 2018

(54) METHOD FOR PRODUCING A GARMENT FOR WORK WITH EMS/EMG/EGG, AND SUCH A GARMENT

(71) Applicant: Björn WOLTERMANN, Berlin (DE)

(72) Inventor: Björn WOLTERMANN, Berlin (DE)

(21) Appl. No.: 15/772,905

(22) PCT Filed: Nov. 2, 2016
(86) PCT No.: PCT/DE2016/100516

§ 371 Date: May 2, 2018

(30) Foreign Application Priority Data
Nov. 3, 2015 (DE) 10 2015 118 837.6

(51) Int. Cl. A61B 5/00; A61B 5/0408; A61N 1/04

(52) U.S. Cl. CPC A61B 5/6804; A61B 5/04085; A61B 5/0492; A61N 1/0484; A61N 1/0452

(57) ABSTRACT

The invention relates to a method for producing a garment (1), and to a garment which is worn on the human body and which is suitable for conducting electrical signals to and/or from the human body. The method comprises the following steps: spreading out a supporting structure (100) which has at least two garment components (110) which are partially coupled by means of at least one partial coupling (120); positioning a prefabricated functional structure (200) or producing a functional structure (200) which has the function of transferring the electrical signals from and/or to the human body in a spatial relationship to the supporting structure (100) in such a way that, when the functional structure (200) crosses a boundary between at least two garment components (110), the functional structure (200) is positioned on the partial coupling (120) between the garment components (110), coupling the supporting structure (100) to the functional structure (200), finishing the garment (1) by means of final coupling of the garment components (110) to each other.

### PRIOR ART

The invention relates to a method for producing a garment which covers parts of the human body in order to work by means of electrical muscle stimulation (EMS), electromyography (EMG), electrocardiography (ECG) or other similar methods. In all of the explanations below in the document, EMS is adopted as the exemplary application. All properties and descriptions apply similarly to EMG, ECG and all similar methods.

The invention furthermore also relates to an EMS garment which covers parts of the human body in order to work by means of EMS.

The use of EMS devices in order to work in physiotherapy and, in very recent times, also in the spheres of sports and fitness, is a growing industry. In addition to the many advantages for the customer or the user of such EMS devices, at the same time, however, a series of new technical challenges emerge therefrom which the user has to overcome.

The garment known in current use consists of a plurality of components. Each part has lines and electrodes installed therein which are designated for the body part which is covered by the part. On occasion, the athlete has to put on said lines and electrodes separately in order then to connect them to a central monitoring unit or, in even more complicated fashion, first of all has to connect the parts to one another. This generally does not happen without the assistance of further individuals.

Production methods for current EMS garments consist of numerous steps. The supporting structure of current suits is sewn together from more than 100 components. The textile and silver-containing electrode surfaces are subsequently sewn onto the regions provided for this purpose. With each wash, the silver-containing textile electrodes lose a portion of the silver and therefore of their conductivity. Alternatively, Velcro pads which later receive bulky and exchangeable electrodes made from textile or plastics are sewn in. In both cases, the electrodes are connected via a classic cable harness which, for its part, likewise consists of 30 or more individual parts and is fastened to the electrodes by means of press studs. Said cable harness, and also the stuck-on electrodes have to be removed in order to clean the garment. Mistakes are moreover possible both during the initial assembly and during the repeated assembly of the garment. It is attempted to minimize said mistakes by means of color codings of the cables and press studs.

First attempts to integrate the lines directly into the garment operate with individual connections per electrode, said connections being sewn successively to the electrode and to the contact surface. In this case, the number of components is not substantially reduced in comparison to the external cable harness. Numerous error sources arise because of the high proportion of manual work at the manufacturer of the current suits. Also, there are some reservations about whether these internal cable harnesses can cope with frequent washing operations, and this greatly restricts the durability among other things, because of the multiplicity of electrical contact points between individual components.

Attempts to integrate the lines with silver-containing threads directly into the fabric of the garment likewise have difficulties with durability, since the silver is gradually washed out. In particular in the case of suits worn directly on the body, washing after each training session is imperative.

Furthermore, fabrics containing silver and nanosilver are questionable for reasons of environmental pollution and are already prohibited in many countries. Furthermore, working steps are necessary to insulate the knitted-in conductor strips from the body. These working steps present further error sources.

Furthermore, all of the known textile electrode solutions have the disadvantage that they have to be provided with a liquid or gel-like medium in order to produce the conductivity to the body. This is also true of all the solutions in which, for hygiene reasons, underwear is worn under the actual EMS garment. One solution provides a conductive rubber which is applied to the textile electrodes. Said rubber ensures that the electrodes do not have to be moistened. Since, however, the rubber does not protect the fabric from liquid media and detergents, this solution does not eliminate the problem of the silver being washed out.

The main focus of this invention is the use of an EMS garment in the spheres of sport and fitness, in which it is necessary for such an EMS garment to be capable of being produced favorably with high quality, is resistant to wear, is comfortable to wear and can be donned by the athlete independently, simply and with repeating accuracy. The invention is based on the problem of how the manufacturer and supplier will improve and develop an EMS garment in order to achieve these aims.

### DISCLOSURE OF THE INVENTION

The invention is based on the object of a method for producing an EMS garment which is resistant to wear, i.e. can be worn and rewashed frequently, can be put on alone and is comfortable to wear, is highly efficient on the muscular system without having to be additionally moistened, by means of good transmission of the electrical signals from a monitoring or control device via a terminal, via an electrically conductive structure, to the electrodes and from the electrodes to the human skin, and from the human skin to the monitoring or control device, and can be produced efficiently with advantageous, but high-quality and simultaneously environmentally protective materials and at maximum speed in as few steps as possible and with as few individual parts as possible.

This object is achieved according to the invention by a method as claimed in claim 1 and by a garment as claimed in claim 20. According thereto, a first component which is referred to as a supporting structure and comprises at least one premanufactured garment component of the EMS garment is spread out. In particular, at least two garment components can be at least partially precoupled to each other, for example by partial sewing together, adhesive bonding, pressing or by other methods suitable for this purpose, in order to make the correct spreading out simpler. The spreading out is undertaken in such a manner that all of the EMS garment surfaces which are provided for a second component, which is referred to as a functional structure which has the function of transmitting the electrical signals from and/or to the human body, can be reached from at least one side.

In a further step, the functional structure is spatially arranged on the supporting structure, or the supporting structure on the functional structure, in such a manner that, when parts of the functional structure cross the boundaries between two garment components of the supporting structure, the parts of the functional structure are positioned precisely on the partial couplings between the garment components of the supporting structure.

In a further step, the functional structure and the supporting structure are coupled by the use of thermal means, pressure, adhesives or other methods.

In a further step, the EMS garment is completely coupled by sewing together, adhesive bonding, pressing or other suitable methods for this purpose at the end, and since the partial couplings are not repeated and the functional structure has been precisely positioned on the partial couplings which have already been produced, said functional structure is not damaged by the complete coupling of the EMS garment.

The functional structure can be produced separately or directly on the supporting structure, and the steps can be carried out in any desired advantageous sequence.

Alternatively, the functional structure can be produced on and/or partially in the supporting structure. In this case, the positioning of the functional structure in the spatial relationship with respect to the supporting structure takes place simultaneously with the coupling of the functional and the supporting structure.

For this purpose, it is provided that the boundaries between the garment components are defined as lines where the garment components meet and where the latter, entirely coupled together, produce a garment, wherein the partial couplings between the garment components are preferably carried out only on the boundaries where the garment components meet when they are to be spread out as a supporting structure. Furthermore, the partial couplings at said boundaries will couple only a part of the boundary that is preferably at least as wide as the width of the functional structure which will cross these points.

For this purpose, it is provided according to the invention that all of the parts of the functional structure take up a position in the garment in order to be able to transmit the electrical signals to and from the body parts designated for them.

By this means, it is possible in an advantageous manner according to the invention for added value to be generated for the customer or the athlete by the invention. Furthermore, it is advantageously possible according to the invention that, by means of this novel method for production, the finished EMS garment is more cost-effective, lighter, longer-lasting and more precise, which increases the useful value of the EMS garment.

For this purpose, it is provided according to the invention that the functional structure comprises at least one terminal, at least one electrode which can transmit the electrical signals to the body and can receive signals from the body, at least one conducting structure which transmits the signals between the electrodes and the terminal, and at least one insulating structure which insulates at least part of the conducting structure, electrodes and terminal from the body.

For this purpose, it is provided according to the invention that at least one conducting structure is produced in a further step by coupling at least one premanufactured line consisting, for example, of a cable or wire, or by casting or injection molding conducting material in a premanufactured die, or by cutting the conducting structure out of a sheet of conducting material, or by 2D/3D printing methods or pressing methods or injection molding methods or plotting, or by another suitable method for this purpose, or a combination thereof.

According to the invention, it is possible to produce the electrodes and the terminal as part of the conducting structure from the conducting material in this step.

For this purpose, it is provided according to the invention that at least one insulating structure is produced in a further step by casting an insulating material in a premanufactured die on the conducting structure, as a result of which the two structures are coupled, or is cast separately in the die, or by cutting out the insulating structure out of a sheet of insulating material, or by 2D/3D printing methods or pressing methods or injection molding methods or plotting, or by another suitable method for this purpose, or a combination thereof, wherein the insulating structure at least completely covers the conducting structure and the terminal, and, in order to ensure that said conducting structure and terminal are well insulated from the body, the covering can be far over the edge of said components, but at least partially does not cover the electrodes, wherein, in order to protect the electrodes from wear, covers the edges of the electrodes, but at the same time leaves the surface of the electrodes free in order to permit direct contact with the skin.

For this purpose, it is provided according to the invention that, in a further step, the insulating structure and the conducting structure have been spread out on each other and are to be coupled by the use of thermal means, pressure, adhesives, casting or other methods if this has not yet taken place during the casting in the die, as a result of which the conducting structure can be functionally split into electrodes, lines and terminals if these have been produced with or as part of the conducting structure in one step.

For this purpose, it is provided according to the invention that, in a further step, the insulating structure and the conducting structure are to be spread out on each other and are to be coupled by the use of thermal means, pressure, adhesives, casting or other methods if this has not yet taken place during the casting in the die, as a result of which the conducting structure can be functionally split into electrodes, lines and terminals if these have been produced with or as part of the conducting structure in one step.

For this purpose, it is provided according to the invention that the electrodes and the terminal are coupled to the conducting structure in a further step if they have been produced separately, wherein the electrodes and the terminal can be produced separately from each other using the same method as for the production of the conducting structure.

For this purpose, it is provided according to the invention that the coupling of the garment components can be achieved by sewing, adhesive bonding, pressure, welding, heating or another method suitable for this purpose, or a combination thereof.

For this purpose, it is provided according to the invention that the conducting and insulating materials are produced from elastic compounds, such as, for example, silicones, in order to assist the elasticity of the garment.

For this purpose, it is provided according to the invention that, in a further step, the conducting material is produced by mixing or connection between an elastic compound and an electrically conducting material.

For this purpose, it is provided according to the invention that the elastic compound is a silicone and the electrically conducting material is a carbon.

For this purpose, it is provided according to the invention that, in a further step, the functional structure is tested with regard to its function prior to the coupling to the supporting structure.

For this purpose, it is provided according to the invention that the lines which lead from the terminals to the electrodes and are intended for mirrored electrodes in each case on the left and right of the human body are designed in such a manner that electrical resistances which do not differ from each other by more than 50% are produced for the two lines in order to keep the results of muscle stimulation identical on both sides and in order not to falsify signals which are received by the body.

For this purpose, it is provided according to the invention that the lines, the conducting structure and the insulating structure are produced in mixed form or jointly in a wave shape, zigzag, spiral or other suitable geometrical arrangements in order to assist the elasticity of the garment.

According to the invention, it is possible for this purpose, in a further implementation, for the EMS garment to consist of two main components, wherein one component, the supporting structure, consists of at least one garment component and the other component, the functional structure, consists of functional parts, for electrically relevant parts in the functional structure to be connected and tested in the one method step and, in a further method step, for the garment components of the supporting structure of the EMS garment to already be at least partially coupled to one another, but for the EMS garment not yet to have been completely coupled, and therefore it is possible, in a further method step, to spread out the supporting structure flat in order to expose all of the surfaces at which the supporting structure is intended to be coupled to the functional structure, and in order to provide a surface over which the functional structure, which does not have to be coupled again, can be laid in order, after the coupling of the supporting and functional structures of the EMS garment, to couple the garment components at the end, advantageously without damaging the functional structure, as a result of which the EMS garment has at the end been produced.

However, it is moreover preferred according to the invention for the EMS garment to be manufactured from at least one garment component which will normally cover the posterior and the legs and preferably consists of a single piece of textile or another material suitable for the human skin, and also from two further garment components, for example sleeve parts, one per arm.

By this means, it is possible in an advantageous manner according to the invention to couple all of the garment components at least partially to one another, wherein the at least partially coupled garment components form a flat structure, wherein this assists the stability of the supporting structure during the coupling to the functional structure, and in order to produce a homogeneous surface which does not have to be coupled again at these points, for example by sewing, which could damage the functional structure.

According to the invention, in a further advantageous individual step, a complete, premanufactured, functional structure is matched to and positioned on the spread-out supporting structure.

It is furthermore preferred according to the invention for the functional structure to be premanufactured from an elastic conducting compound, wherein the compound can be mixed or cast together or brought together by another method, wherein it is then advantageously spread out, cut out, cast or injection molded or, with the aid of another method, brought into the precise shape and size which is necessary for an EMS garment, depending on the size needed for the individual person.

It is therefore advantageously possible according to the invention for a particularly flexible functional structure to be produced in order to assist this, and for the compound to consist of an elastic carrier material, which is an insulating material, and a conducting material, said structure correspondingly assisting the flexibility of the two materials because of further geometrical arrangements.

According to the invention, the shape of the electrodes and conducting structures is selected in such a manner that they can take part in the movements of the body and therefore the garment in a manner as free as possible from resistance by, for example, the electrode surfaces obtaining a high degree of flexibility with the aid of incisions, notches, wave shapes or other advantageous geometrical shapes.

According to the invention, it is furthermore preferred for the elastic carrier material to be a silicone, but also any other elastic material, wherein it would be advantageous to use skin-compatible materials, and wherein the conducting material is a carbon powder or an electric line which can be rigid, but has been designed in an extendable form, for example spring or wave shape, or by means of conducting, liquid materials which can harden, or else consists of an elastic carrier material on which the intended shapes and properties are produced with the aid of electrically conductive coatings such that the conducting structure restricts the flexibility of the carrier material as little as possible.

In an advantageous manner, the functional structure as a whole can be checked and tested for damage or faults in a step prior to the coupling to the supporting structure.

It is thereby advantageously possible according to the invention for particularly precise and rapid production of the functional structure to take place, said structure requiring few resources and being durable and highly elastic.

According to the invention, in a further advantageous individual step, the functional structure is coupled to the spread-out, open supporting structure, advantageously in one step, by the use of pressure, heat, adhesive, other methods or a combination thereof, wherein the functional structure is permanently and stably connected to the supporting structure.

According to the invention, in a further advantageous step, the functional structure is produced from an insulating structure which is matched precisely to a conducting structure, as a result of which the lines are advantageously insulated from the body, and therefore the electrical signals are not weakened on the way to and from the electrode.

According to a further advantageous embodiment, the functional structure is formed integrally. This means in particular that the functional structure does not have any connecting or coupling points, such as, for example, soldering points or plug-in elements, by means of which two or more parts of the functional structure are connected to one another retrospectively. For this purpose, it can be provided, for example, that the functional structure is cast as a whole or piece by piece from suitable materials.

According to the invention, it is furthermore also preferred here for the insulating structure to be composed of a non-conducting material, for example silicone, but it can also be composed of any other elastic material, wherein it would be more advantageous to use a skin-compatible material, wherein the material can then be advantageously spread out, cut out, cast or injection molded directly onto the conducting structure, or is brought with the aid of another method into the precise shape and size which is necessary for an EMS garment depending on the size needed for the individual person, wherein the insulating structure is formed in such a manner that the latter does not cover all parts of the conducting structure, but rather only covers those which should not have any contact with the skin; furthermore, the insulating structure can reach for a distance over the sides of the parts to be insulated, for the sake of safety.

According to the invention, in a further advantageous step, the insulating structure is coupled to the conducting structure and thereby to the garment components of the EMS garment in an advantageous manner in one step by the use of pressure, heat, adhesive, other methods or a combination thereof, as a result of which the insulating structure is connected permanently and stably to the EMS garment components and to the conducting structure, as a result of which advantageously only those parts of the electrodes which are provided to have direct contact with the skin can be.

Furthermore, a computer program product can be provided which controls a device for transmitting signals, wherein the device can be attached to the EMS garment directly at the terminal or by means of a terminal.

Furthermore, the subject matter of the present invention is a garment which is produced by the method according to an embodiment described herein.

Further details, features and advantages of the invention emerge from the drawings and from the description below of preferred embodiments with reference to the drawings. The drawings here merely illustrate exemplary embodiments of the invention which do not restrict the essential inventive concept.

### BRIEF DESCRIPTION OF THE DRAWINGS

FIG. 1 schematically shows a garment according to the present invention.

FIG. 2 schematically shows a supporting structure which, according to the invention, is partially coupled.

FIG. 3 schematically shows a functional structure according to the invention.

FIG. 4 schematically shows the construction of a die.

FIGS. 5a-5e schematically show possible embodiments of the electrodes.

FIGS. 6a-6f schematically show possible embodiments of the lines.

FIG. 7 schematically shows the construction of a functional structure.

### EMBODIMENTS OF THE INVENTION

The present invention is described with reference to particular embodiments and with reference to the attached drawings, wherein, however, the invention is not limited to these embodiments and to these drawings, but rather is determined by the patent claims. The drawings should not be understood as limiting. Certain elements may be illustrated in the drawings in enlarged or exaggerated form and not true to scale, for illustrative purposes.

Unless specifically indicated differently, the use of an indefinite or definite article with respect to a word in the single, for example “a” or “the”, also includes the plurality of such a word. The designations “first” and “second” and so on in the description and in the claims are used for differentiating between similar elements or for differentiating identical elements and not necessarily for the description of a temporal or other sequence. The terms used in such a manner should basically be considered as interchangeable under corresponding conditions.

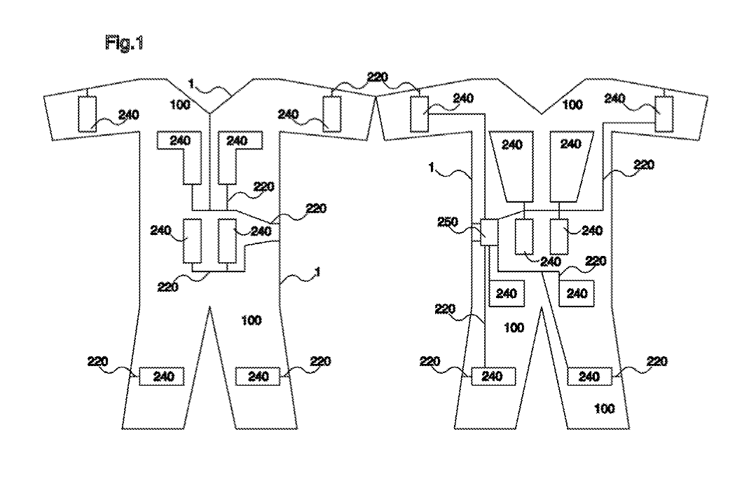

In FIG. 1, a garment (1) composed of, for example, textiles, which covers the human body is shown in such a manner that the front and the rear side of the garment (1) can be seen from the inside. The garment (1) has a supporting structure (100) and a functional structure (200), wherein the functional structure (200) is at least partially attached on the inner side of the garment (1) in order to bring parts thereof in contact with the human skin.

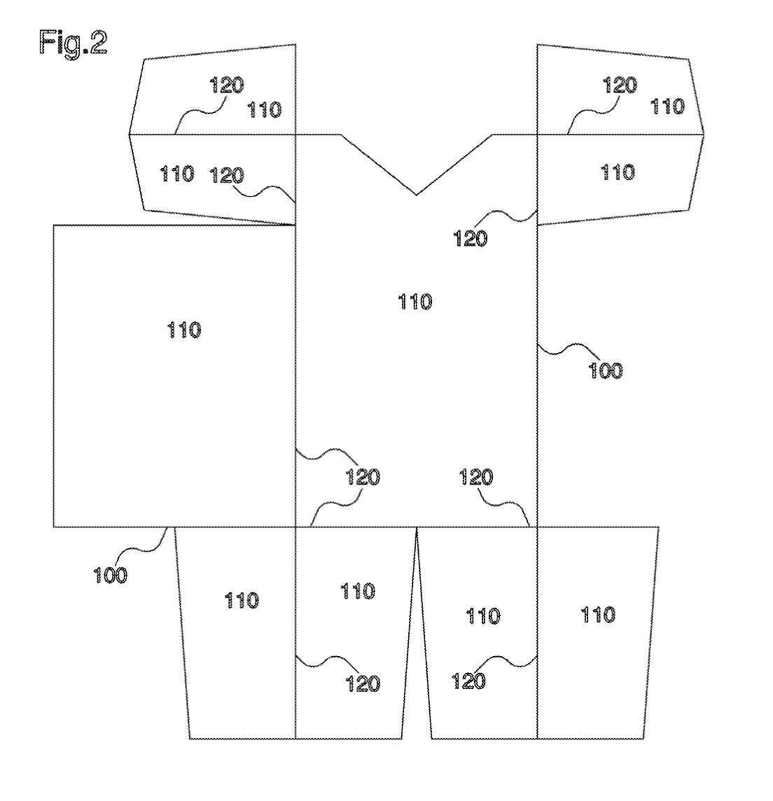

The supporting structure (100) according to FIG. 2 is constructed from a material suitable for wearing against the human skin. The material can be directly knitted from one piece according to a provided pattern, or produced by another suitable method, or can consist of one or more garment components (110) which are at least partially coupled to one another by means of, for example, sewing or adhesive bonding or another suitable method in order to have a garment (1) as the result at the end. The garment (1) covers at least part of the body, but can also be a full body garment. The garment (1) is conceived to permit freedom of movement and not only for this reason is constructed precisely to the height of different people and, after being donned, is intended to lie closely against the skin. The garment components (110) are only partially coupled to one another, thus making it possible for the supporting structure (100) to be spread out flat.

The boundary between the garment components (110) run in the finished garment (1) far beyond the boundaries shown in FIG. 2 and should also be seen as the lines where the garment components meet and where the latter, entirely coupled together, result in a garment. The partial couplings (120) produced by the partial coupling are at least as wide as the width of that part of the functional structure (200) which is laid over said partial coupling (120). FIG. 2 schematically shows a supporting structure (100) in which the garment components (110) have been partially coupled to each other at part of the boundary. If more than the partial coupling were to take place, it would no longer be possible to spread out the supporting structure (100) flat. The partial couplings (120) which are shown are precisely as wide here as the boundary part, illustrated in FIG. 2, between the garment components (110), wherein said partial couplings can be much smaller and can take place only on one part of said boundary part.

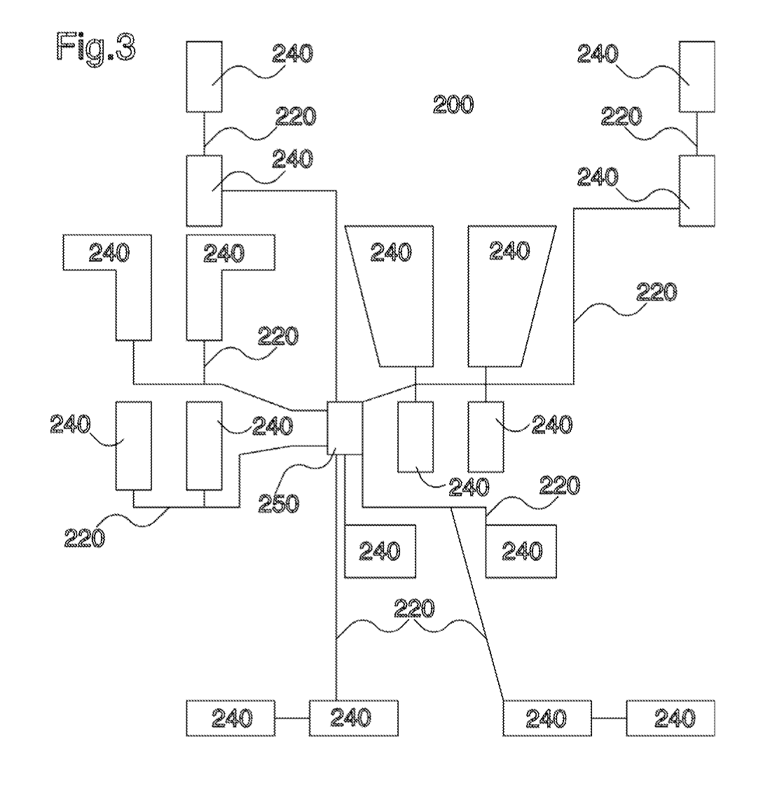

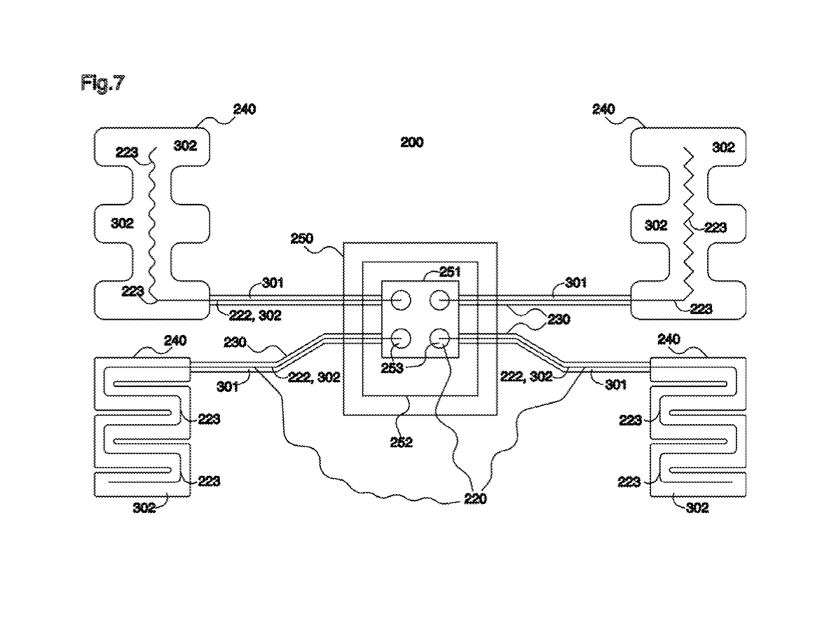

The functional structure (200) according to FIG. 3 or 7 includes at least one terminal (250), at least one conducting structure (220) which is connected to the terminal (250), at least one insulating structure (230) which insulates the conducting structure (220) at at least one point against the body and ensures that said conducting structure does not come into contact with the human skin at at least one point, and at least one electrode (240) which is connected to the conducting structure (220) and which transmits the electrical signals to and from the human skin, wherein FIG. 3 does not illustrate the insulating structure (230), in order to improve the clarity.

The conducting structure (220) according to FIG. 3 or 7 serves for the purpose of transmitting the electrical signals between the terminal (250) and the electrodes (240). In a preferred embodiment, the conducting structure (220) is attached as a whole on the inner side of the garment (1), but can also be entirely or partially attached on the outer side of the garment (1).

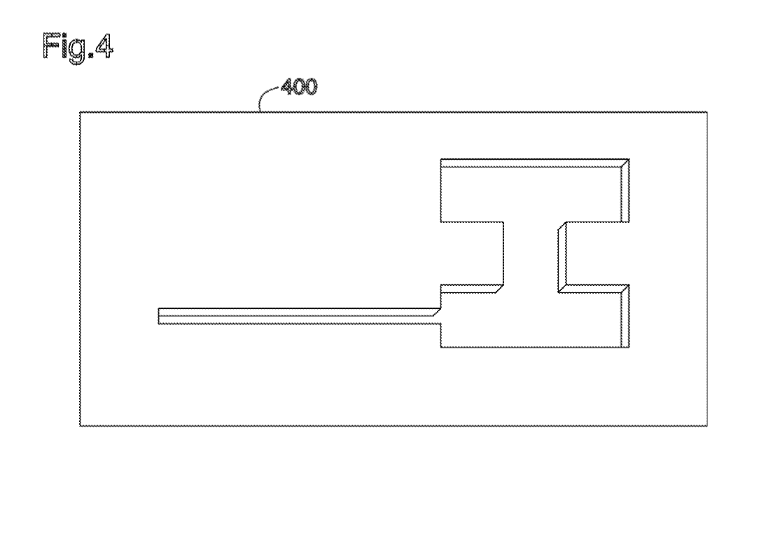

In a preferred embodiment, the conducting structure (220) is produced by means of an injection molding or casting method, in which, in a first step, a casting mold or a die (400) according to FIG. 4 is produced. A plurality of and different dies can be produced depending on the size and shape of the garment (1).

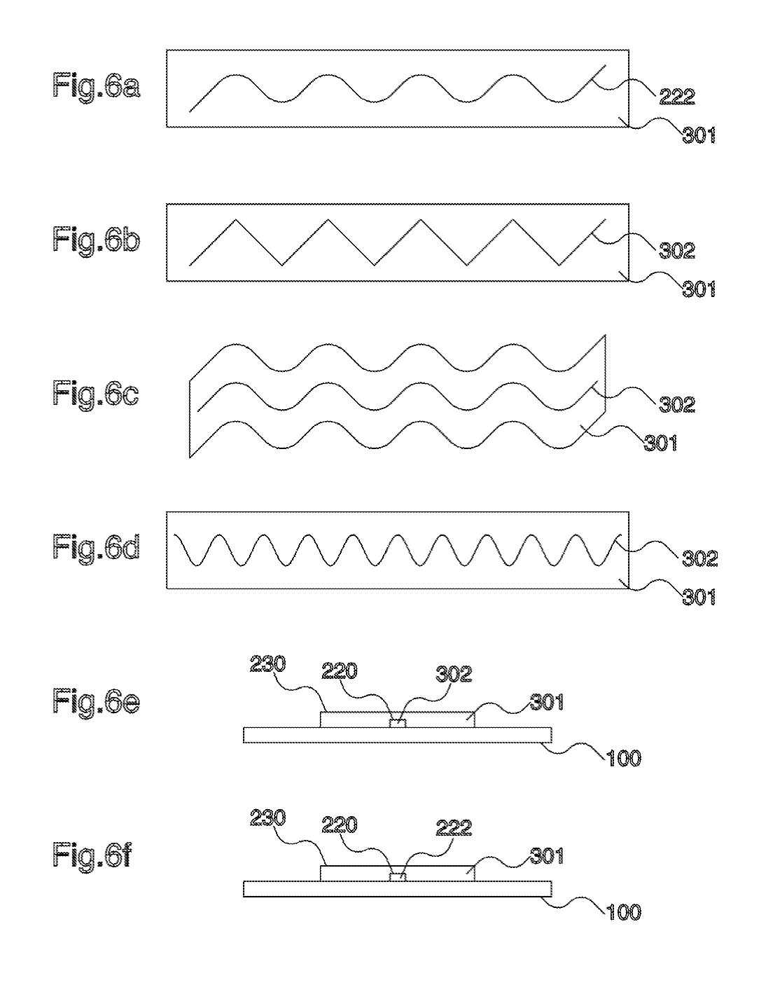

In a preferred embodiment, the conducting structure (220) is produced by laying at least one line (222) into the die (400), wherein each line (222) takes up its designated position, and by casting an insulating material (301) into the die (400), said material then hardening. The hardening of the cast insulating material (301) produces the insulating structure (230). A cross section thereof is illustrated in FIG. 6f. In a further embodiment, without using a line (222), a conducting material (302) can be cast into the die (400), said conducting material hardening and allowing the desired conducting structure (220) to be produced. If, in a further step, an insulating material (301) is cast thereon, the insulating structure (230) is also produced here directly in the die (400). A cross section thereof is illustrated in FIG. 6e.

According to FIGS. 6a to 6d, the lines (222) can be placed in various ways into the die (400) in order to increase the flexibility of the conducting structure (220) and to improve the flexibility of the functional and supporting structures (100, 200). FIG. 6a shows a wavy structure, FIG. 6b shows a zigzag-like structure and FIG. 6d shows a spiral structure of the line (222). Said structures ensure that the line (222) is not damaged when the garment (1) is stretched. Furthermore, a wavy structure is also illustrated for the insulating material (301) in FIG. 6c, wherein said structure can also be used for the conducting material (302), which structure is achieved by an appropriate configuration of the die (400). Any desired combination of said structures and also other structures which increase the flexibility can be used.

In a further embodiment, a combination between the conducting material (302) and the use of electric lines (222) for the optimum transmission of the signals are possible. An insulating structure (230), for example consisting of the insulating material (301) but also of other possible insulating materials, can be cast here into the die (400) around the conducting material (302) in order to insulate the conducting material (302) from the human skin and further influencing factors which could interfere with the transmission of the electrical signals. In the cases in which the conducting structure (220) has not been sufficiently insulated beforehand, an insulating structure (230) is fitted on the conducting structure (220).

In a further embodiment possibility, the lines (222) are spread out into the required position on a surface and the materials (301,302) are cast thereon as already described, but without using a die (400). After the materials (301, 302) have hardened, the conducting structure (220) is cut out by, for example, laser, waterjet, tungsten carbide blades or other methods suitable for this purpose. In the cases in which the conducting structure (220) has not been sufficiently insulated beforehand, an insulating structure (230) is fitted on the cut-out conducting structure (220). The insulating structure (230) can also be produced here by casting and/or cutting, wherein a combination of the methods used is possible for both methods.

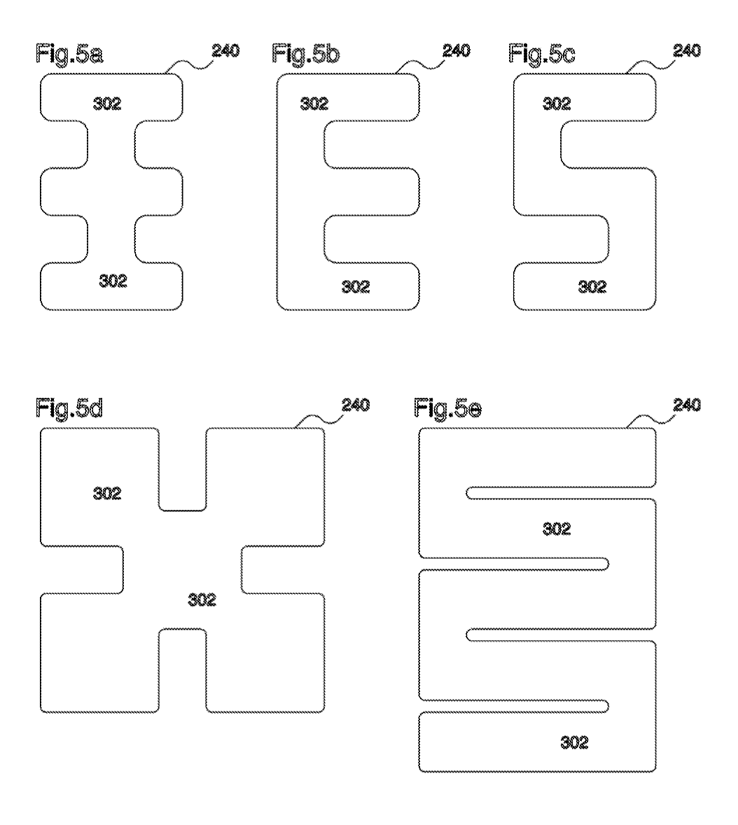

In a further step, the electrodes (240) are produced according to FIGS. 5a-5e. The shapes of the electrodes (240), as illustrated in FIGS. 5a to 5e, have been determined depending on various factors. An electrode (240) intended for use in an EMS garment (1) should be elastic, flexible and stretchable such that said electrode is matched precisely to the skin of the body shape in order thereby to obtain as large a contact surface as possible so as to transmit the signals to and from the skin and furthermore to the muscles as efficiently as possible. The shape of the electrode has to be simultaneously matched to the shape of the muscle to be stimulated. That is to say in practice that, for example, an electrode shape (240) of FIG. 5a fits better to a group of muscles around which it circles, for example in the case of an extremity. By contrast, the electrode (240) from FIG. 5d is more suitable for stimulating a flat group of muscles, for example the chest muscles, or an individual muscle at a certain point.

The preferred method for producing the electrodes (240) is also selected depending on the preferred method for producing the conducting structure (220). If the conducting structure (220) is produced in a die (400) by the use of electrical lines (222) and an insulating material (301), the line ends (223) are configured in such a manner that they are placed precisely wherever an electrode (240) is intended to be produced. At the same time, the insulating material (301) is cast in such a manner that the line ends (223) are not covered. Alternatively, the line ends (223) can also be freed from the insulating material (301) in an intermediate step. In a further step, the electrodes (240) are cast by the conducting material (302) being cast onto the line ends (223) in the die, wherein the die (400) predetermines the desired shape of the electrodes (240). By means of this rapid method, the materials (301) and (302) are connected to one another seamlessly and permanently. However, if necessary, recourse can also be made to other means, such as, for example, adhesives or local heating, in order to permanently ensure the coupling. Furthermore, the electrodes (240) can be produced individually from preferred materials, such as, for example, conducting material (302), silver or other materials and retrospectively coupled to the line ends (223). Adhesives or local heating can be used in order to permanently ensure the coupling to the conducting structure (220). Care should be taken here to ensure that the materials and methods used do not impair the conductivity.

If the conducting structure (220) is produced by using lines (222), an insulating material (301) and cutting out, the line ends (223) are configured in such a manner that they are placed precisely wherever an electrode (240) is intended to be produced. At the same time, the insulating material (301) is cast in such a manner that the line ends (223) are not covered. Alternatively, the line ends (223) can also be freed from the insulating material (301) in an intermediate step. In a further step, the electrodes (240) are cast by the conducting material (302) being cast onto the line ends (223) and subsequently being cut out into the desired electrode shape. By means of this rapid method, the materials (301) and (302) are connected to one another seamlessly and permanently. However, if necessary, recourse can also be made to other means, such as, for example, adhesives or local heating in order to permanently ensure the coupling. Furthermore, the electrodes (240) can be produced individually from preferred materials, such as, for example, conducting material (302), silver or other materials, and subsequently coupled on to the line ends (223). Adhesives or local heating can be used in order to permanently ensure the coupling to the conducting structure (220).

If the conducting structure (220) is produced by casting the conducting material (302) with or without lines (222) in a die (400) or by casting and cutting out, the electrodes (240) are cast together in one step in the die (400) as part of the conducting structure (220), or are cast together and cut out together. In a further step, a layer of insulating material (301) is cast around the conducting structure (220), but not around the electrodes (240). Alternatively, an insulating structure (230) which is precast and/or precut from insulating material (301) can be coupled to the conducting structure (220) such that the latter does not cover or insulate the electrodes (240). By means of these two methods, the electrodes (240) are functionally produced without the subsequent casting of the conducting material (302), or without the electrodes (240) having to be produced separately and coupled subsequently to the conducting structure (220).

According to FIG. 3 or 7, the terminal (250) is the component which produces the connection between the functional structure (200) and an external monitoring unit. The terminal (250) is preferably configured in such a manner that it forms a bridge between the inner side of the garment (1), on which said terminal is coupled to the conducting structure (220), and the outer side of the garment (1), on which the external monitoring unit is ideally located. The terminal (250) consists of a printed circuit board (251) to which line ends (223) are connected. Before the casting and/or cutting, the printed circuit board (251) can be placed into a premanufactured socket-like mold (252) and connected to the line ends (223) by means of contact points (253). The printed circuit board (251) together with the socket-like mold (252) is brought with the conducting structure (220) into the required position in the die (400) and then covered with the insulating material (301), as a result of which the printed circuit board is insulated by the insulating material (301) on the side with the line ends (223) and sealed in the socket-like mold (252). This gives rise to the terminal (250) as part of the functional structure (200).

A cable bundle which connects the garment (1) to a monitoring unit, in the form of a computer with a computer program product, can be connected to the socket-like mold (252) by means of a plug. In a preferred embodiment of the invention, instead of a plug with a cable bundle, use is made of a transceiver unit which receives the signals of the monitoring unit by radio, e.g. Bluetooth or WLAN (Wi-Fi) or by another transmitting method suitable for this purpose, and then converts said signals into electrical signals for the electrodes (240). Conversely, electrical signals from the electrodes (240) are converted by the transceiver unit into radio signals and transmitted to the monitoring unit. In a further embodiment, the monitoring unit is reduced in size in such a manner that the latter fits completely into the socket-like mold (252) in the garment (1).

In a further embodiment, as described above, the conducting structure (220) is cast directly from conducting material (302) into a die (400), or without a die (400) is cut out of a sheet of conducting material (302). In these embodiments, the terminal (250) which consists of the printed circuit board (251) and the socket-like mold (252) is coupled to the line ends (223) by means of contact points (253) after the formation of the lines (222). In a further step, the conducting structure (220) is sealed with insulating material (301). In a further embodiment, the terminal (250) can be positioned directly in the die (400) before the casting and the lines can be cast onto the contact points (253). By this means, a coupling step is omitted.

With the aid of the described steps, or any desired combination thereof, a functional structure (200) and a supporting structure (100) are produced. Said two structures can be investigated and tested separately from each other for faults and functionality.

In a further step, the supporting structure (100) and the functional structure (200) are combined with each other. This can be referred to as in the automobile industry as the “marriage”. The supporting structure (100) is spread out without creases on a surface and the functional structure (200) is placed thereon, wherein the process can also be carried out in reverse. The two structures (100, 200) are then coupled permanently to each other by means of adhesive or heat or pressure or by means of another suitable method or a combination thereof. It is important to note that all of the parts of the functional structure (100) are placed precisely onto the partial couplings (120) between the garment components (110). Furthermore, it is important for the position of the partial couplings (120) to be selected in such a manner that the functional structure (200) can optimally reach all regions of the garment (1).

In a further step, all of the partial couplings (120) of the supporting structure (100) are coupled at the end, as a result of which the garment (1) is produced. Owing to the fact that all of the parts of the functional structure (200) have already been placed onto the partial couplings (120) in a previous step, said structure is not damaged by the subsequent coupling of the garment components (110).

The previously described method steps result in the production of the garment (1) for work with EMS, EMG or ECG and all similar methods.

### LIST OF REFERENCE SIGNS

garment (1)
supporting structure (100)
garment component (110)
partial coupling (120)
functional structure (200)
conducting structure (220)
line (222)
line end (223)
insulating structure (230)
electrode (240)
terminal (250)
printed circuit board (251)
socket-like mold (252)
contact point (253)
insulating material (301)
conducting material (302)
die (400)

1. A method for producing a garment (1) which is worn on the human body and is suitable for transmitting electrical signals to and/or from the human body in a conductive manner and which comprises the following steps:
spreading out a supporting structure (100) which has at least two garment components (110) which are partially coupled by means of at least one partial coupling (120);
positioning a premanufactured functional structure (200) or producing a functional structure (200), which has the function of transmitting the electrical signals from and/or to the human body, in a spatial relationship with respect to the supporting structure (100) in such a manner that, when the functional structure (200) crosses a boundary between at least two garment components (110), the functional structure (200) is positioned on the partial coupling (120) between the garment components (110),
coupling the supporting structure (100) to the functional structure (200),
finishing the garment (1) by means of coupling the garment components (110) to one another at the end.

2. The method as claimed in claim 1, characterized in that the supporting structure (100) is positioned or produced and in the spatial relationship with respect to the functional structure (100, 200) in such a manner that at least part of the functional structure (200) lies in the garment (1) such that said part can transmit the signals to and from at least one body part.

3. The method as claimed in claim 1, characterized in that the functional structure (200) comprises at least one electrode (240), at least one terminal (250), at least one conducting structure (220) and at least one insulating structure (230).

4. The method as claimed in claim 3, characterized in that the conducting structure (220) is produced by coupling at least one premanufactured line (222)
or by casting or injecting conducting material (302) into a die (400),
or by cutting the conducting structure (220) out of a sheet of conducting material (302),
or by a 2D/3D printing method with conducting material (302),
or another method suitable for this purpose,
or a combination thereof,
in at least one line harness (221).

5. The method as claimed in claim 3, characterized in that the insulating structure (230) is produced by
casting insulating material (301) into the die (400) onto the conducting structure (220),
or is cast separately into the die (400),
or by means of cutting of the insulating structure (230) out of a sheet of insulating material (301),
or by 2D/3D printing methods with insulating material (301),
or another method suitable for this purpose,
or a combination thereof.

6. The method as claimed in claim 3, characterized in that the insulating structure (230) completely covers the conducting structure (220) and the terminal (250) but at least partially does not cover the electrodes (240).

7. The method as claimed in claim 3, characterized in that the electrodes (240) and/or the terminal (250) as part of the conducting structure (220) are/is produced from the conducting material (302) in one step.

8. The method as claimed in claim 3, characterized in that the electrodes (240) and/or the terminal (250) are/is produced separately from the conducting material (302) using the same method as the conducting structure (220).

9. The method as claimed in claim 2, characterized in that the electrodes (240) and/or the terminal (250) are/is coupled to the conducting structure (220) in a further step.

10. The method as claimed in claim 2, characterized in that the conducting structure (220) and the insulating structure (230) are spread out on each other and coupled to each other.

11. The method as claimed in claim 7, characterized in that, by means of the coupling of the conducting structure (220) to the insulating structure (230), the electrodes (240) are functionally separated from the conducting structure (220).

12. The method as claimed in claim 1, characterized in that the coupling is achieved by sewing, adhesive bonding, pressure, welding, heating or a combination thereof.

13. The method as claimed in claim 1, characterized in that the conducting and insulating materials (301, 302) are produced from at least one elastic compound, such as, for example, silicone.

14. The method as claimed in claim 1, characterized in that the conducting material (302) is a bond between at least one elastic compound and at least one electrically conducting material.

15. The method as claimed in claim 1, characterized in that the electrically conducting material is carbon.

16. The method as claimed in claim 1, characterized in that, in a further step, the functional structure (200) is tested for damage or faults prior to the coupling to the supporting structure (100).

17. The method as claimed in claim 1, characterized in that the lines (222) which lead from the terminal (250) to the electrodes (240) and are intended for mirrored electrodes (240) in each case for the left and right side of the human body are designed in such a manner that the resistance for the two lines (222) does not differ by more than 50%.

18. The method as claimed in claim 1, characterized in that the lines (222), the conducting structure (220) and the insulating structure (230) are produced in mixed form or jointly in a wavy, zigzag-shaped, or spiral arrangement or in further geometrical arrangements in order to assist the elasticity of the garment (1).

19. The method as claimed in claim 1, characterized in that the functional structure is formed integrally.

20. A garment (1) which is worn on the human body and is suitable for transmitting electrical signals to and/or from the human body in a conducting manner, comprising:
a supporting structure (100) which has at least two garment components (110) which are partially coupled by means of at least one partial coupling (120);
a functional structure (200) which has the function of transmitting the electrical signals from and/or to the human body, and is arranged in a spatial relationship with respect to the supporting structure (100) in such a manner that, when the functional structure (200) crosses a boundary between at least two garment components (110), the functional structure (200) is positioned on the partial coupling (120) between the garment components (110).

21. A garment (1) produced by means of a method as claimed in claim 1.
